(19) United States
(12) Patent Application Publication
(10) Pub. No.: US 2024/0409884 A1
(43) Pub. Date: Dec. 12, 2024

(54) REMEDIATION MATERIAL FOR HEAVY METAL POLLUTION OF SOIL, AND PREPARATION METHOD AND USE THEREOF

(71) Applicant: Shandong Academy of Agricultural Sciences, Jinan City (CN)

(72) Inventors: Yanqin WANG, Jinan City (CN); Zhaodong LIU, Jinan City (CN); Longyun FU, Jinan City (CN); Yan LI, Jinan City (CN); Hanlin ZHANG, Jinan City (CN); Ziwen ZHONG, Jinan City (CN); Chengchang HU, Jinan City (CN); Luji BO, Jinan City (CN); Yongping JING, Jinan City (CN)

(21) Appl. No.: 18/470,058

(22) Filed: Sep. 19, 2023

(30) Foreign Application Priority Data
Jun. 9, 2023 (CN) 2023106867902

(51) Int. Cl. C12N 1/20; B09C 1/10

(52) U.S. Cl. CPC C12N 1/205; B09C 1/10; B09C 2101/00; C12R 2001/125

(57) ABSTRACT

The present disclosure relates to the technical field of remediation for heavy metal pollution of soil, in particular to a remediation material for heavy metal pollution of soil, and a preparation method and use thereof. In the present disclosure, the remediation material is obtained by compounding specific Bacillus subtilis ASD05 with straw biochar. The remediation material has a desirable remediation effect on heavy metal pollution, especially on cadmium-contaminated soil, such that an effective cadmium content in the soil is reduced by 40% to 60%. Meanwhile, a raw material of the straw biochar is straw, which has a wide range of sources and a low cost, and effectively avoids the waste of crop straw resources, thereby improving a utilization rate of the straw resources and reducing environmental pollution.

### CROSS-REFERENCE TO RELATED APPLICATION

This patent application claims priority to Chinese Patent Application No. 2023106867902 filed with the China National Intellectual Property Administration on Jun. 9, 2023, the disclosure of which is incorporated by reference herein, in its entirety, as part of the present application.

### TECHNICAL FIELD

The present disclosure relates to the technical field of remediation for heavy metal pollution of soil, in particular to a remediation material for heavy metal pollution of soil, and a preparation method and use thereof.

### BACKGROUND

Facility tomato and cucumber are the main vegetable varieties planted. As main by-products of the tomato and cucumber cultivation, wastes such as tomato straw and cucumber straw are generally dumped in the field, easily spreading diseases and causing waste of resources. In addition, manure organic fertilizers have been used in facility vegetable greenhouses for many years. It is possible that the soil has a high background value of cadmium, or the facility vegetable production area is close to a mining area. As a result, continuous cropping for not less than 5 years leads to cadmium pollution in the soil. Plants can easily absorb Cd from Cd-contaminated soils and accumulate in their edible parts. The Cd directly or indirectly accumulates in the human body through the food chain, causing damages to the human body.

There are many measures to control Cd exceeding the standard in crops. For polluted soil, soil remediation is mainly conducted to achieve the safe production. A more effective method for remediation and treatment of Cd pollution is to apply some soil passivation materials, including lime, fly ash, hydroxyapatite, and organic fertilizers, thereby increasing soil pH and soil adsorption sites, reducing Cd activity in the soil, and controlling root absorption of crops. However, the existing remediation methods show high remediation cost and poor remediation effect, and cannot solve the waste of crop straw resources.

### SUMMARY

In order to solve the above problems, the present disclosure provides a remediation material for heavy metal pollution of soil, and a preparation method and use thereof. In the remediation material of the present disclosure, crop straws are prepared into straw biochar, and compounded with a suitable Bacillus subtilis strain. This not only solves the waste of crop straw resources, but also shows a desirable remediation effect on Cd-contaminated soil, with a low remediation cost.

To achieve the above objective, the present disclosure provides the following technical solutions:

The present disclosure provides a remediation material for heavy metal pollution of soil, including the following raw materials: straw biochar and Bacillus subtilis ASD05; where the Bacillus subtilis ASD05 is deposited in the China General Microbiological Culture Collection Center (CGMCC), with a deposit number of CGMCC No. 17807.

Preferably, a preparation method of the straw biochar includes pyrolytic carbonization; and

the pyrolytic carbonization is conducted at a pyrolytic carbonization temperature of 400° C. to 600° C. for 1.5 h to 2.5 h, and the pyrolytic carbonization temperature is obtained by heating at 8° C./min to 15° C./min.

In one embodiment, a raw material of the straw biochar is one or more selected from the group consisting of tomato straw, cucumber straw, corn straw, eggplant straw, and garlic straw.

In one embodiment, the particle size of the raw material for preparing the straw biochar is not more than 0.15 mm.

The present disclosure further provides a preparation method of the remediation material, including the following steps:
mixing a seed solution of the Bacillus subtilis ASD05, the straw biochar, and a medium, and culturing the Bacillus subtilis ASD05 to a logarithmic growth phase to obtain a mixture; where the seed solution of the Bacillus subtilis ASD05, the straw biochar, and the medium are at a dosage ratio of (1-3) mL:(2-3) g:100 mL; the seed solution of the Bacillus subtilis ASD05 has a concentration of viable bacteria at (1-9)×10⁸ cells/mL; and the medium includes the following components by concentration: 10 g/L of peptone, 5 g/L of a yeast powder, and 5 g/L of sodium chloride; and
subjecting the mixture to centrifugation, and discarding a resulting supernatant to obtain the remediation material.

In one embodiment, the preparation method further includes the following step before the mixture is subjected to centrifugation: shaking the mixture at 37° C. and 160 r/min for 12 h.

In one embodiment, the preparation method further includes the following step after the supernatant is discarded: subjecting a precipitate obtained after the supernatant is discarded to washing and centrifugation 3 times with a 1 wt. % sodium chloride solution.

The present disclosure further provides use of the remediation material or a remediation material prepared by the preparation method in remediation for heavy metal pollution of soil.

In one embodiment, the heavy metal includes cadmium.

The present disclosure further provides a method for remediating heavy metal pollution of soil, including the following steps:
applying a remediation material to soil to be remediated; where the remediation material is applied at 120 kg/mu to 260 kg/mu; and the remediation material is the remediation material described above or a remediation material prepared by the preparation method described above.

### Beneficial Effects:

The present disclosure provides a remediation material for heavy metal pollution of soil, including the following raw materials: straw biochar and Bacillus subtilis ASD05; where the Bacillus subtilis ASD05 is deposited in the China General Microbiological Culture Collection Center (CGMCC), with a deposit number of CGMCC No. 17807. In the present disclosure, the remediation material is obtained by compounding specific Bacillus subtilis ASD05 with straw biochar. The remediation material has a desirable remediation effect on heavy metal pollution, especially on cadmium-contaminated soil, such that an effective cadmium content in the soil is reduced by 40% to 60%. Meanwhile, a raw material of the straw biochar is straw, which has a wide range of sources and a low cost, and effectively avoids the waste of crop straw resources, thereby improving a utilization rate of the straw resources and reducing environmental pollution.

### Deposit of Biological Material

The Bacillus subtilis ASD05 strain was deposited in the CGMCC located at NO. 1 West Beichen Road, Chaoyang District, Beijing 100101, China on May 15, 2019, with a deposit number of CGMCC No. 17807.

### BRIEF DESCRIPTION OF THE DRAWINGS

To illustrate the examples of the present disclosure or the technical solutions in the prior art more clearly, the accompanying drawings required in the examples will be briefly introduced below.

FIG. 1 shows a scanning electron microscopy (SEM) image of straw biochar prepared in step 2) of Example 1; and

FIG. 2 shows an SEM image of the remediation material prepared in Example 1.

### DETAILED DESCRIPTION OF THE EMBODIMENTS

The present disclosure provides a remediation material for heavy metal pollution of soil, including the following raw materials: straw biochar and Bacillus subtilis ASD05; where the Bacillus subtilis ASD05 is deposited in the China General Microbiological Culture Collection Center (CGMCC), with a deposit number of CGMCC No. 17807.

In the present disclosure, a preparation method of the straw biochar preferably includes pyrolytic carbonization. The pyrolytic carbonization is conducted at preferably 400° C. to 600° C., more preferably at 400° C. to 500° C., more preferably at 400° C. for preferably 1.5 h to 2.5 h, more preferably 2 h. The pyrolytic carbonization temperature is obtained by heating at preferably 8° C./min to 15° C./min, more preferably at 9° C./min to 12° C./min, and more preferably at 10° C./min. A time for conducting the pyrolytic carbonization is a time after the pyrolytic carbonization temperature is achieved.

In the present disclosure, a device for preparing the straw biochar preferably includes a tubular furnace. The tubular furnace is preferably a 1,200° C. open-type tubular furnace with a model number of OTF-1200X purchased from Hefei Kejing Material Technology Co., Ltd.

In the present disclosure, the straw biochar prepared by a suitable preparation method has a stable structure, a huge surface area, and porous properties, thereby showing strong stability and adsorption. The straw biochar has a desirable effect on remediating cadmium pollution in soil, especially the biochar prepared under a pyrolytic carbonization temperature of 400° C. has the best remediation effect.

In the present disclosure, a raw material of the straw biochar is preferably one or more selected from the group consisting of tomato straw, cucumber straw, corn straw, eggplant straw, and garlic straw; more preferably the tomato straw and the cucumber straw; and the tomato straw and the cucumber straw are at a mass ratio of preferably (1-2):1, more preferably 1:1.

In the present disclosure, the raw material of the straw biochar has a particle size of preferably less than or equal to 0.15 mm. More preferably, the straw is pulverized and passed through a 100-mesh sieve, and obtained undersize is the raw material for the preparation of the straw biochar.

In the present disclosure, the remediation material is obtained by compounding specific Bacillus subtilis ASD05 with straw biochar. The remediation material has a desirable remediation effect on heavy metal pollution, especially on cadmium-contaminated soil, such that an effective cadmium content in the soil is reduced by 40% to 60%. Meanwhile, a raw material of the straw biochar is straw, which has a wide range of sources and a low cost, and effectively avoids the waste of crop straw resources, thereby improving a utilization rate of the straw resources and reducing environmental pollution.

The present disclosure further provides a preparation method of the remediation material, including the following steps:
mixing a seed solution of the Bacillus subtilis ASD05, the straw biochar, and a medium, and culturing the Bacillus subtilis ASD05 to a logarithmic growth phase to obtain a mixture; where the seed solution of the Bacillus subtilis ASD05, the straw biochar, and the medium are at a dosage ratio of (1-3) mL:(2-3) g:100 mL, preferably 2.5 mL; 2.5 g:100 mL; the seed solution of the Bacillus subtilis ASD05 has a concentration of viable bacteria at (1-9)×10⁸ cells/mL; and the medium includes the following components by concentration: 10 g/L of peptone, 5 g/L of a yeast powder, and 5 g/L of sodium chloride; and
subjecting the mixture to centrifugation, and discarding a resulting supernatant to obtain the remediation material.

In the present disclosure, the seed solution of the Bacillus subtilis ASD05 has a concentration of viable bacteria at (1-9)×10⁸ cells/mL, preferably (1-5)×10⁸ cells/mL, and more preferably 1×10⁸ cells/mL. A process for preparing the seed solution of the Bacillus subtilis ASD05 preferably includes: subjecting a Bacillus subtilis ASD05 strain to activation culture in a beef extract peptone medium for 24 h to obtain a culture solution; and

inoculating the culture solution into a fresh beef extract peptone medium to allow enrichment culture for 24 h to obtain the seed solution of the Bacillus subtilis ASD05.

In the present disclosure, the activation culture is conducted at preferably 37° C. and 160 r/min; the culture solution and the fresh beef extract peptone medium are at a volume ratio of preferably 2:100; and the enrichment culture is conducted at preferably 37° C. and 160 r/min.

In the present disclosure, after the seed solution of the Bacillus subtilis ASD05 is obtained, the seed solution of the Bacillus subtilis ASD05, the straw biochar, and the medium are mixed, and the Bacillus subtilis ASD05 is cultured to a logarithmic growth phase to obtain the mixture.

In the present disclosure, the seed solution of the Bacillus subtilis ASD05, the straw biochar, and the medium are at a dosage ratio of (1-3) mL:(2-3) g:100 mL, more preferably 2.5 mL:2.5 g:100 mL. The medium includes the following components by concentration: 10 g/L of peptone, 5 g/L of a yeast powder, and 5 g/L of sodium chloride. The medium has a pH value of preferably 7.

In the present disclosure, the culturing is conducted at preferably 37° C. and 160 r/min.

In the present disclosure, the Bacillus subtilis ASD05 and the straw biochar are co-cultivated in the same medium, such that the Bacillus subtilis can be better evenly loaded on the straw biochar, which is beneficial for the Bacillus subtilis and the biochar to work together during the remediation of cadmium-contaminated soil.

In the present disclosure, after the mixture is obtained, the mixture is preferably shaken at 37° C. and 160 r/min for 12 h.

In the present disclosure, the shaking culture of the mixture under suitable conditions can further make the Bacillus subtilis evenly loaded on the straw biochar, such that they can be combined more tightly.

In the present disclosure, after the mixture is shaken for 12 h, a new mixture shaken for 12 h is centrifuged, and a resulting supernatant is discarded to obtain a primary remediation material. The centrifugation is conducted at preferably 3,000 r/min for 8 min to 12 min, more preferably 10 min.

In the present disclosure, after the primary remediation material is obtained, a precipitate obtained after discarding the supernatant (that is, the primary remediation material) is preferably subjected to washing and centrifugation 3 times with a 1 wt. % sodium chloride solution to obtain a solid precipitate.

In the present disclosure, the washing preferably includes rinsing; each rinsing is conducted for preferably 2 min.

In the present disclosure, centrifugation is preferably conducted after each washing to obtain a precipitate for the next washing or to obtain the solid precipitate; and the centrifugation is preferably conducted at 3,000 r/min for 10 min.

In the present disclosure, the medium and unloaded microorganisms on a surface of the solid precipitate can be cleaned by washing with an appropriate concentration of the sodium chloride solution to obtain a pure remediation material.

In the present disclosure, after the solid precipitate is obtained, the solid precipitate is preferably sub-packaged, and then freeze-dried to obtain the remediation material.

In the present disclosure, the sub-packaging is preferably conducted under aseptic conditions for preferably within 1 h to 2 h to avoid contamination by bacteria.

In the present disclosure, a process of the freeze-drying preferably includes: pre-freezing for 3 h, and drying at −84° C. for 48 h at a room pressure of 0.04 mbar after a temperature reaches −20° C. to −35° C.

In the present disclosure, a freezer for the freeze-drying is preferably purchased from Chongqing MaiRuiSi Experimental Equipment Co., Ltd.

In the present disclosure, the Bacillus subtilis is immobilized on the straw biochar by a suitable culture method, which can provide a protective barrier for the Bacillus subtilis. This avoids the Bacillus subtilis being adversely affected by competition with indigenous microorganisms, excessive concentrations of pollutants, and unfavorable pH and temperature. The prepared remediation material can improve a soil structure and change bioavailability of heavy metal cadmium through the dual actions of biochar and microorganisms when applied to cadmium-contaminated soil. In this way, a water and fertilizer use efficiency and a nutrient content in the soil can be enhanced, and the cadmium-contaminated soil can be remediated.

The present disclosure further provides use of the remediation material or a remediation material prepared by the preparation method in remediation for heavy metal pollution of soil.

In the present disclosure, the heavy metal preferably includes cadmium. The heavy metal-contaminated soil to be remediated preferably includes one or more of the following types: soil that has been applied manure organic fertilizer for not less than 5 years, soil with cadmium content of greater than 0.6 mg/kg, soil within 1 km from a mining area, and soil with continuous cropping for not less than 5 years, and further preferably includes soil with a pH value of 6.5 to 7.5 and a cadmium content of (0.6-1.0) mg/kg.

The present disclosure further provides a method for remediating heavy metal pollution of soil, including the following steps:

applying a remediation material to soil to be remediated; where the remediation material is the remediation material described above or a remediation material prepared by the preparation method described above.

In the present disclosure, the remediation material is applied at 120 kg/mu to 260 kg/mu, preferably 200 kg/mu to 260 kg/mu, more preferably 200 kg/mu. Applying suitable remediation materials has a desirable remediation effect on cadmium-contaminated soil, reducing the effective cadmium content in the soil by 40% to 60%.

In the present disclosure, an application method preferably includes: spreading the remediation material on the soil to be treated, plowing and watering to a maximum field capacity, and conducting soil moisture retention for 10 d to 15 d. The soil moisture retention is conducted for preferably 10 d to 15 d, more preferably 11 d to 14 d, and even more preferably 12 d. The soil moisture retention is conducted to a moisture content of preferably 60% to 80%.

In the present disclosure, the plowing can fully mix the remediation material and the soil; a proper time of the soil moisture retention can make both the biochar and the microorganisms in the remediation material exert their maximum effects.

In order to further illustrate the present disclosure, the remediation material for heavy metal pollution of soil, and the preparation method and the use thereof provided in the present disclosure are described in detail below with reference to examples, but the examples should not be interpreted as a limitation to the protection scope of the present disclosure.

### Example 1

A preparation method of a remediation material for heavy metal pollution of soil included the following steps:

1) Tomato straw and cucumber straw were collected from facilities, plastic film and slings on the straw were removed, the two kinds of straw were separately air-dried and chopped into pieces of 3 cm to 5 cm, pulverized with a pulverizer, ground, and passed through a 100-mesh sieve to obtain under-sieved materials, which were tomato straw powder and cucumber straw powder.

2) Certain amounts of the tomato straw powder and the cucumber straw powder obtained in step 1) were filled into an alumina tube, and heated to a pyrolysis temperature of 400° C. at 10° C./min in a tubular furnace (purchased from Hefei Kejing Material Technology Co., Ltd., a 1,200° C. open-type tubular furnace with a model of OTF-1200X) to allow 2 h of pyrolysis (the 2 h of pyrolysis referred to heating to 400° C., followed by heat preservation at 400° C. for 2 h), so as to obtain straw biochar. The tomato straw powder and the cucumber straw powder were at a mass ratio of 1:1.

3) Preparation of a seed solution of Bacillus subtilis ASD05: a Bacillus subtilis ASD05 strain was subjected to activation culture in a beef extract peptone medium for 24 h to obtain a culture solution; where the Bacillus subtilis ASD05 was deposited in the CGMCC with a deposit number of CGMCC No. 17807; and the activation culture was conducted at 37° C. and 160 r/min; and

The culture solution was inoculated into a fresh beef extract peptone medium to allow enrichment culture for 24 h to obtain the seed solution of the Bacillus subtilis ASD05; where the culture solution and the fresh beef extract peptone medium were at a volume ratio of 2:100; the enrichment culture was conducted at 37° C. and 160 r/min; and the seed solution of the Bacillus subtilis ASD05 had a concentration of viable bacteria at 1×10⁸ cells/mL.

4) A medium was prepared, the straw biochar obtained in step 2) was put into the medium, autoclaved at 121° C. for 20 min, the seed solution of the Bacillus subtilis ASD05 obtained in step 3) was inoculated, and the Bacillus subtilis ASD05 was cultured to a logarithmic growth phase at 37° C. and 160 r/min to obtain a mixed solution; where the medium included the following components by concentration: 10 g/L of peptone, 5 g/L of a yeast powder, and 5 g/L of sodium chloride: the medium had a pH value of 7; and the seed solution of the Bacillus subtilis ASD05, the straw biochar, and the medium were at a dosage ratio of 2.5 mL:2.5 g:100 mL.

5) The mixed solution obtained in step 4) was shaken at 37° C. and 160 r/min for 12 h, then centrifuged (3,000 r/min) for 10 min, and a resulting supernatant was discarded: the remaining lower precipitate was washed with a 1 wt. % normal saline, centrifuged (3,000 r/min) and washed repeatedly 3 times to obtain a solid, which was an immobilized microbial precipitate.

6) The immobilized microbial precipitate was sub-packaged into glass bottles, pre-frozen, and freeze-dried with a freeze dryer to obtain a solid, which was the remediation material for heavy metal pollution of soil.

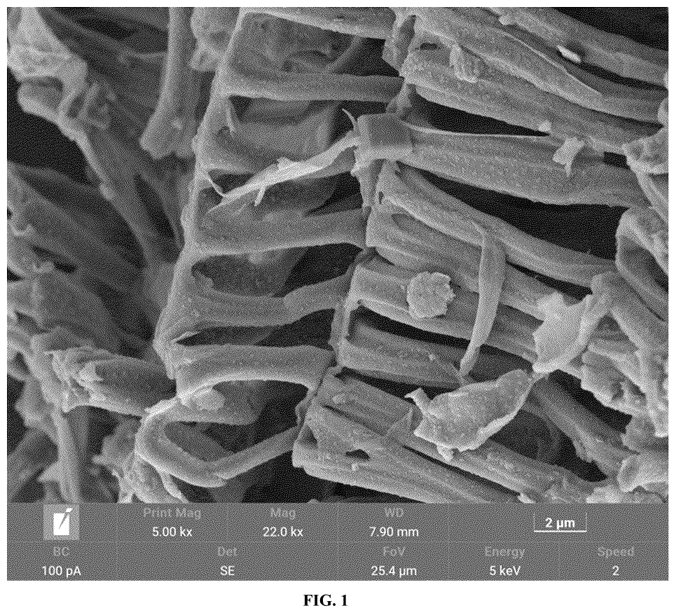

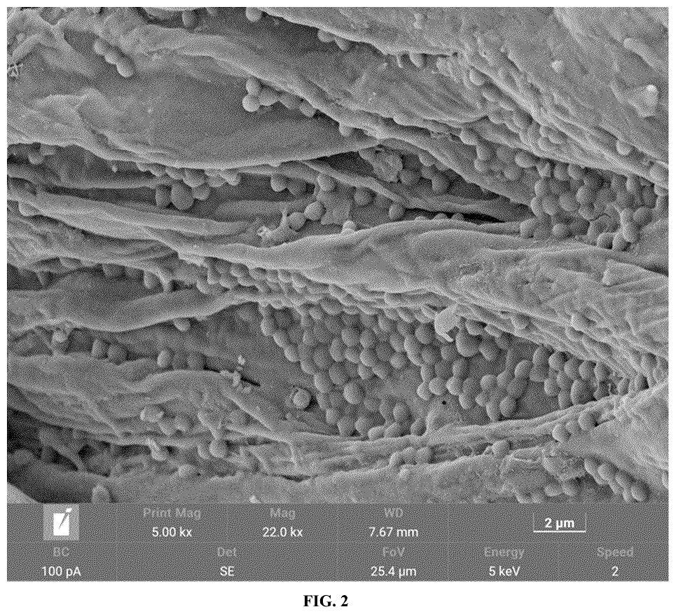

The changes of surface morphology of the straw biochar prepared in step 2) and the biochar of the remediation material obtained in step 6) were observed by SEM (TESCAN MIRA LMS, Czech Republic). The sample particles from the biochar were evenly placed on a loading plate, and sprayed with gold, and the loading plate was put into a sample box for detection, and the morphology of the particles was observed on-machine. Results were shown in FIG. 1 and FIG. 2.

As shown in FIG. 1 and FIG. 2, the microorganisms are extremely well loaded on the biochar, indicating that the loading is extremely successful.

### Example 2

This example provided a remediation material similar to that of Example 1, the only difference was that the pyrolysis was conducted at 500° C.

### Example 3

This example provided a remediation material similar to that of Example 1, the only difference was that the pyrolysis was conducted at 600° C.

### Comparative Use Example 1

The specific surface area (SSA), pore size, and pore volume of the remediation materials prepared in Examples 1 to 3 were measured using an ASAP-2460 surface area analyzer (BET, Micromeritics Instrument Corporation), and the results were shown in Table 1.

TABLE 1
Performance comparison of three remediation materials

| Remediation | SSA | Pore size | Pore volume |
|---|---|---|---|
| material | (m²/g) | (nm) | (cm³/g) |
| Example 1 | 0.9832 | 7.3365 | 0.003548 |
| Example 2 | 0.7273 | 5.1254 | 0.002241 |
| Example 3 | 0.4126 | 3.2224 | 0.001387 |

As shown in Table 1, the SSA, pore size, and pore volume of the remediation material of Example 1 each are better than those of the remediation materials of Examples 2 and 3.

### Example 4

A method for remediating soil cadmium pollution included the following steps:

The remediation material prepared in Example 1 was spread in facility soil at 120 kg/mu. There was cadmium pollution in the soil due to continuous cropping of vegetables in a greenhouse for not less than 5 years: the soil was determined to have a pH of 7.2 and a cadmium content of 0.9 mg/kg. The soil was fully plowed, watered to a maximum field capacity, and subjected to soil moisture retention for 12 d, and facility vegetables were planted for 2 years and 4 seasons.

### Example 5

This example provided a method similar to that of Example 4, the only difference was that the remediation material was applied at 200 kg/mu.

### Example 6

This example provided a method similar to that of Example 4, the only difference was that the remediation material was applied at 220 kg/mu.

### Example 7

This example provided a method similar to that of Example 4, the only difference was that the remediation material was applied at 260 kg/mu.

### Example 8

This example provided a method similar to that of Example 5, the only difference was that the remediation material prepared in Example 1 was replaced with the remediation material prepared in Example 2.

### Example 9

This example provided a method similar to that of Example 5, the only difference was that the remediation material prepared in Example 1 was replaced with the remediation material prepared in Example 3.

### Comparative Example 1

This comparative example provided a remediation material similar to that of Example 1, except that the Bacillus subtilis ASD05 in step 3) was replaced with a Bacillus licheniformis strain, to prepare a seed solution of the Bacillus licheniformis; meanwhile, the seed solution of the Bacillus subtilis ASD05 in step 4) was replaced with the seed solution of the Bacillus licheniformis. The Bacillus licheniformis strain was purchased from the CGMCC, with a deposit number of CGMCC No. 1.807.

### Comparative Example 2

This comparative example provided a remediation material similar to that of Example 1, except that the Bacillus subtilis ASD05 in step 3) was replaced with a Bacillus mucilaginosus strain, to prepare a seed solution of the Bacillus mucilaginosus; meanwhile, the seed solution of the Bacillus subtilis ASD05 in step 4) was replaced with the seed solution of the Bacillus mucilaginosus. The Bacillus mucilaginosus strain was purchased from the China Center of Industrial Culture Collection (CICC), with a deposit number of CICC 20667.

### Comparative Example 3

This example provided a method similar to that of Example 5, the only difference was that the remediation material prepared in Example 1 was replaced with the remediation material prepared in Comparative Example 1.

### Comparative Example 4

This example provided a method similar to that of Example 5, the only difference was that the remediation material prepared in Example 1 was replaced with the remediation material prepared in Comparative Example 2.

### Comparative Example 5

This example provided a method similar to that of Example 5, the only difference was that the remediation material prepared in Example 1 was replaced with the straw biochar prepared in step 2 of Example 1.

### Comparative Use Example 2

A cadmium content of 0 cm to 20 cm of soil before and after remediation in Examples 4 to 9 and Comparative Examples 3 to 5 was measured by GB/T 17141-1997 “Soil quality—Determination of lead, cadmium—Graphite furnace atomic absorption spectrophotometry”, and a cadmium removal efficiency of the soil was calculated. Soil cadmium passivation rate=(available cadmium content in 0-20 cm of soil before remediation-available cadmium content in 0-20 cm of soil after remediation)/available cadmium content in 0-20 cm of soil before remediation×100%. A crop planted in each example or comparative example was tomato. An available cadmium content in the soil was measured before planting, and the available cadmium content in the soil was measured after the tomato was harvested. Each example or comparative example was set with 3 parallel repeated measurement experiments, and the measurement results were shown in Table 2.

TABLE 2
Effect comparison of different examples and comparative examples

| | | Available cadmium | Available cadmium | Soil |
|---|---|---|---|---|
| | | content in 0-20 cm | content in 0-20 cm | cadmium |
| | | of soil before | of soil after | passivation |
| Group | Difference from Example 5 | remediation (mg/kg) | remediation (mg/kg) | rate (%) |
| Example 4 | Remediation material applied at 120 | 0.49 | 0.29 | 40.8 |
| | kg/mu | | | |
| Example 5 | — | 0.49 | 0.24 | 51.0 |
| Example 6 | Remediation material applied at 220 | 0.49 | 0.24 | 51.0 |
| | kg/mu | | | |
| Example 7 | Remediation material applied at 260 | 0.49 | 0.23 | 53.1 |
| | kg/mu | | | |
| Example 8 | Pyrolysis of biochar at 500° C. | 0.49 | 0.30 | 38.8 |
| Example 9 | Pyrolysis of biochar at 600° C. | 0.49 | 0.32 | 34.7 |
| Comparative | Bacillus licheniformis as loaded | 0.49 | 0.31 | 36.7 |
| Example 3 | microorganism | | | |
| Comparative | Bacillus mucilaginosus as loaded | 0.49 | 0.29 | 40.8 |
| Example 4 | microorganism | | | |
| Comparative | Biochar unloaded with Bacillus subtilis | 0.49 | 0.33 | 32.7 |
| Example 5 | ASD05 | | | |

As shown in Table 2, it is seen from the comparison of effects in Examples 4 to 7 that the remediation material of the present disclosure shows a same effect at 220 kg/mu and 200 kg/mu, and the effect improvement is not obvious at 260 kg/mu. Considering cost and benefit, the soil remediation material is best used at 200 kg/mu. From the comparison of effects in Examples 5, 8, and 9, it is seen that the biochar prepared at a pyrolysis temperature of 400° C. has the highest passivation rate of cadmium in soil. From the comparison of effects in Example 5 and Comparative Examples 3 to 5, it is seen that the biochar without immobilizing microorganisms has the worst passivation effect on cadmium in the soil, and the available cadmium content in the soil is only decreased by 32.7%; while the Bacillus subtilis ASD05 has the best remediation effect, and the available cadmium content in the soil decreases by 51.0%. From the comparison of effects in Example 4 and Comparative Example 4, it is seen that when the microorganisms loaded on biochar are not suitable, the remediation effect can be improved by increasing an application amount of remediation material, but a cost might increase significantly. From the comparison of effects in Examples 8 and 9, and Comparative Examples 3 and 4, it is seen that when the microorganisms loaded on biochar are not suitable, the remediation effect can be improved by adjusting a pyrolysis temperature for preparing the biochar. This indicates that an appropriate pyrolysis temperature is dominant relative to the remediation effect of loaded microorganisms on soil.

In summary, in the remediation material of the present disclosure, crop straws are prepared into straw biochar, and compounded with a suitable Bacillus subtilis strain. This not only solves the waste of crop straw resources, but also shows a desirable remediation effect on Cd-contaminated soil, with a low remediation cost.

Although the above example has described the present disclosure in detail, it is only a part of, not all of, the examples of the present disclosure. Other examples may also be obtained by persons based on the example without creative efforts, and all of these examples shall fall within the protection scope of the present disclosure.

What is claimed is:

1. A remediation material for heavy metal pollution of soil, the remediation material comprising the following raw materials:
straw biochar; and
Bacillus subtilis ASD05;
wherein the Bacillus subtilis ASD05 is deposited in the China General Microbiological Culture Collection Center (CGMCC), with a deposit number of CGMCC No. 17807.

2. The remediation material according to claim 1, wherein a method for preparing the straw biochar comprises pyrolytic carbonization; and
the pyrolytic carbonization is conducted at a pyrolytic carbonization temperature of 400° C. to 600° C. for 1.5 h to 2.5 h, and the pyrolytic carbonization temperature is obtained by heating at 8° C./min to 15° C./min.

3. The remediation material according to claim 1, wherein a raw material of the straw biochar is one or more selected from the group consisting of tomato straw, cucumber straw, corn straw, eggplant straw, and garlic straw.

4. The remediation material according to claim 1, wherein the particle size of the raw material for preparing the straw biochar is not more than 0.15 mm.

5. The remediation material according to claim 4, wherein a raw material of the straw biochar is one or more selected from the group consisting of tomato straw, cucumber straw, corn straw, eggplant straw, and garlic straw.

6. A method for preparing the remediation material according to claim 1, the method comprising:
mixing a seed solution of the Bacillus subtilis ASD05, the straw biochar, and a medium, and culturing the Bacillus subtilis ASD05 to a logarithmic growth phase to obtain a mixture;
wherein the seed solution of the Bacillus subtilis ASD05, the straw biochar, and the medium are at a dosage ratio of (1-3) mL:(2-3) g:100 mL; the seed solution of the Bacillus subtilis ASD05 has a concentration of viable bacteria at (1-9)×10⁸ cells/mL; and the medium comprises the following components by concentration: 10 g/L of peptone, 5 g/L of a yeast powder, and 5 g/L of sodium chloride; and
subjecting the mixture to centrifugation, and discarding a resulting supernatant to obtain the remediation material.

7. The method according to claim 6, wherein the particle size of the raw material for preparing the straw biochar is not more than 0.15 mm.

8. The method according to claim 6, further comprising the following step after the supernatant is discarded: subjecting a precipitate obtained after the supernatant is discarded to washing and centrifugation 3 times with a 1 wt. % sodium chloride solution.

9. The method according to claim 6, wherein a raw material of the straw biochar is one or more selected from the group consisting of tomato straw, cucumber straw, corn straw, eggplant straw, and garlic straw.

10. The method according to claim 6, wherein a method for preparing the straw biochar comprises pyrolytic carbonization; and
the pyrolytic carbonization is conducted at a pyrolytic carbonization temperature of 400° C. to 600° C. for 1.5 h to 2.5 h, and the pyrolytic carbonization temperature is obtained by heating at 8° C./min to 15° C./min.

11. The method according to claim 6, further comprising the following step before the mixture is subjected to centrifugation: shaking the mixture at 37° C. and 160 r/min for 12 h.

12. A method for remediating heavy metal pollution of soil, the method comprising:
applying a remediation material to soil to be remediated;
wherein the remediation material is applied at 120 kg/mu to 260 kg/mu; and the remediation material is the remediation material according to claim 1.

13. The method according to claim 12, wherein the heavy metal comprises cadmium.

14. The method according to claim 12, wherein a method for preparing the straw biochar comprises pyrolytic carbonization; and
the pyrolytic carbonization is conducted at a pyrolytic carbonization temperature of 400° C. to 600° C. for 1.5 h to 2.5 h, and the pyrolytic carbonization temperature is obtained by heating at 8° C./min to 15° C./min.

15. The method according to claim 12, wherein a method for preparing the remediation material, further comprising:
mixing a seed solution of the Bacillus subtilis ASD05, the straw biochar, and a medium, and culturing the Bacillus subtilis ASD05 to a logarithmic growth phase to obtain a mixture;
wherein the seed solution of the Bacillus subtilis ASD05, the straw biochar, and the medium are at a dosage ratio of (1-3) mL:(2-3) g:100 mL; the seed solution of the Bacillus subtilis ASD05 has a concentration of viable bacteria at (1-9)×10⁸ cells/mL; and the medium comprises the following components by concentration: 10 g/L of peptone, 5 g/L of a yeast powder, and 5 g/L of sodium chloride; and
subjecting the mixture to centrifugation, and discarding a resulting supernatant to obtain the remediation material.

16. The method according to claim 12, wherein the particle size of the raw material for preparing the straw biochar is not more than 0.15 mm.

17. The method according to claim 16, the method for preparing the remediation material further comprises the following step before the mixture is subjected to centrifugation:
shaking the mixture at 37° C. and 160 r/min for 12 h.

18. The method according to claim 16, the method for preparing the remediation material further comprises the following step after the supernatant is discarded: subjecting a precipitate obtained after the supernatant is discarded to washing and centrifugation 3 times with a 1 wt. % sodium chloride solution.

19. The method according to claim 12, wherein a raw material of the straw biochar is one or more selected from the group consisting of tomato straw, cucumber straw, corn straw, eggplant straw, and garlic straw.
